(19) United States
(12) Patent Application Publication
(10) Pub. No.: US 2016/0345805 A1
(43) Pub. Date: Dec. 1, 2016

(54) SET OF PRISMS, PRISM HOLDER ARRANGEMENT AND ENDOSCOPE WITH A VARIABLE VIEWING DIRECTION

(71) Applicant: OLYMPUS WINTER & IBE GMBH, Hamburg (DE)

(72) Inventors: Martin WIETERS, Barsbuettel (DE); Jens ROSE, Hamburg (DE); Patrick SCHERR, Alt-Moelln (DE)

(73) Assignee: OLYMPUS WINTER & IBE GMBH, Hamburg (DE)

(21) Appl. No.: 15/233,259

(22) Filed: Aug. 10, 2016

Related U.S. Application Data
(63) Continuation of application No. PCT/EP2015/052406, filed on Feb. 5, 2015.

(30) Foreign Application Priority Data
Feb. 13, 2014 (DE) 102014202669.5

(51) Int. Cl. A61B 1/00; G02B 7/18; G02B 26/08; G02B 23/24; A61B 1/04; G02B 23/02

(52) U.S. Cl. CPC A61B 1/00096; A61B 1/00183; A61B 1/0011; A61B 1/04; G02B 23/02; G02B 26/0816; G02B 23/2423; G02B 7/1805

(57) ABSTRACT

A set of prisms with a variable viewing direction including: three prisms; the first and third prisms each have a light entrance, mirror surface and light exit surfaces, the second prism has a beam path surface having light entrance and exit regions and at least two mirror surfaces, the first prism is pivoted relative to the second prism about an axis of rotation that runs through the mirror surface of the first prism and one of the at least two mirror surfaces of the second prism, and the third prism is immovably arranged with respect to the second prism, and the second prism is sectionally wider than the first and/or third prisms transversally to a beam path such that at least one lateral wider section of the single beam path surface of the second prism extends on at least one side beyond the light entrance surface of the third prism.

### CROSS-REFERENCE TO RELATED APPLICATION

The present application is a continuation of PCT/EP2015/052406 filed on Feb. 5, 2015, which is based upon and claims the benefit to DE 10 2014 202 669.5 filed on Feb. 13, 2014, the entire contents of each of which are incorporated herein by reference.

### BACKGROUND

Field

The present application relates to a set of prisms for an endoscope with a variable viewing direction, comprising a first prism, a second prism and a third prism which together define a beam path passing through the three prisms, wherein the first prism and the third prism each have a light entrance surface, a mirror surface and a light exit surface, and the second prism has a single beam path surface having a light entrance region and a light exit region and at least two mirror surfaces, wherein the first prism can be pivoted with respect to the second prism about an axis of rotation that runs through the mirror surface of the first prism and a first mirror surface of the second prism, and the third prism is arranged in an immovable manner with respect to the second prism. The present application also relates to a prism holder arrangement comprising a set of prisms for an endoscope with a variable viewing direction, and a holder for the set of prisms, as well as a corresponding endoscope with a variable viewing direction.

Prior Art

Endoscopes, such as video endoscopes, in which the light of a surgical field entering at a distal tip of an endoscope shaft of the endoscope is directed through an optical system onto a proximal ocular or one or more images sensors, are known in different designs. Thus, there are endoscopes with a direct view, a so-called 0° viewing direction, or endoscopes with a lateral viewing direction, which have for example a lateral viewing direction of 30°, 45°, 70° or the like differing from the 0° viewing direction. The named degree numbers hereby mean the polar angle between the central viewing axis and the longitudinal axis of the endoscope shaft. There are also endoscopes or respectively video endoscopes with an adjustable lateral viewing direction, in which the viewing angle, i.e. the deviation from the direct view, is adjustable. Besides an adjustment of the viewing angle, i.e. of the deviation from the direct view, the viewing direction, i.e. the azimuth angle, can also be adjusted around the longitudinal axis of the endoscope shaft, in that the endoscope is rotated in its entirety around the longitudinal axis of the endoscope shaft.

European patent application EP 2 369 395 A1 discloses an optical system for a video endoscope in which the viewing angle is changed in that a first prism of a prism group with three prisms arranged distally in the endoscope shaft is rotated, or respectively pivoted, about an axis of rotation that is perpendicular, or respectively transverse to the longitudinal axis of the endoscope shaft. This first prism is also designated a swivel prism. The two other prisms which, together with the first prism, define the optical beam path, are glued to each other and do not rotate conjointly.

The prisms are cut from glass blocks. The width of the respective glass block depends on the size of the beam path. The swivel prism is accommodated in a swivel prism holder and positioned by three contact surfaces. The swivel prism holder itself is rotatably mounted in a mount. In the design and production of corresponding prism groups or sets of prisms, the alignment of the prism relative to its axis of rotation is very important. During rotation, a tumbling of the swivel prism about the axis of rotation can produce significant impairment of the image quality.

The three prisms of the set of prisms are arranged like a chicane. This means that on its side facing the two prisms, the second or middle prism only has a slight amount of space and potential support surface between the two other prisms. In the mount, there is a narrow support bar on which the second prism is placed and aligned. During assembly, the position of the second prism is difficult to guarantee since the narrow support bar functions like a seesaw, and the second prism therefore has a strong tendency to tilt.

### SUMMARY

An object is to guarantee the alignment of the set of prisms, such as the second prism, and to simplify assembly.

Such object can be achieved by a set of prisms for an endoscope with a variable viewing direction, comprising a first prism, a second prism and a third prism which together define a beam path passing through the three prisms, wherein the first prism and the third prism each have a light entrance surface, a mirror surface and a light exit surface, and the second prism has a single beam path surface having a light entrance region and a light exit region and at least two mirror surfaces, wherein the first prism can be pivoted with respect to the second prism about an axis of rotation that runs through the mirror surface of the first prism and a first mirror surface of the second prism, and the third prism is arranged in an immovable manner with respect to the second prism, characterized in that the second prism is configured at least sectionally wider than the first prism and/or the third prism transversally to the course of the beam path, wherein at least one lateral, wider section of the beam path surface of the second prism extends on at least one side beyond the light entrance surface of the third prism.

The present disclosure makes use of the concept that the width of the second prism, i.e., transversally to the central optical axis, or respectively the central beam path, is larger than the optically necessary surface, whereas the two other prisms retain their width and are therefore adapted to the beam path. The second prism therefore extends laterally beyond the third prism and, if applicable, beyond the first prism. The second prism can also extend beyond the third prism and, if applicable, the first prism on both sides. The projecting lateral section subsequently functions as an enlarged support surface in a mount so that the second prism and the third prism, which may be glued to the second prism, do not tilt. The additionally required lateral installation space for the prism group, or respectively set of prisms, serves to securely align to the set of prisms.

The at least one laterally wider section of the beam path surface of the second prism can extend on at least one side beyond the light exit surface of the first prism in a 0° viewing direction of the set of prisms. The 0° viewing direction of the set of prisms is defined by the longitudinal extension of the sequence of the first, second and third prism. The 0° viewing direction is also termed the direct view. Given a first prism with a rectangular surface area, a corner of the rectangular surface area of the first prism can also extend into the wider lateral section of the prism in the event of crossing.

The third prism can have an abutting edge on its side facing the first prism. This abutting edge can serve as a contact edge for aligning the prism group, or respectively the set of prisms, in the mount, providing that the mount has a corresponding complementary contact surface. This can, for example, be arranged on the side of a support bar of the mount.

Before assembly, the third prism can be glued to the second prism. In this context, the alignment of the two prisms relative to each other should be considered, as well as leaving the laterally wider section of the beam path surface of the second prism exposed.

The object is also achieved by a prism holder arrangement comprising the previously-described set of prisms for an endoscope with a variable viewing direction, and a mount for the set of prisms, wherein the mount has at least one support surface for the at least one laterally wider section of the beam path surface of the second prism which, in the longitudinal direction of the endoscope, extends longer than a minimum distance between the first prism and the third prism.

In addition to the set of prisms with the wider second prism, this prism holder arrangement can have the complementary support surface for the wider section of the second prism in the mount. Thus, the contact surface, or respectively mount surface, of the second prism is significantly enlarged in the longitudinal direction of the endoscope in the cooperation between the wider section of the beam path surface of the second prism and the support surface of mount so that, in contrast to the known prior art, a narrow, seesaw-like support surface does not cause tilting, and instead a solid base against tilting is established.

The mount can additionally comprise a support bar with a support bar surface, wherein in a mounted state, a section of the beam path surface of the second prism lies on the support bar surface which is arranged between the first prism and third prism. This further enlarges the support surface, and a further enhancement of tilting resistance is achieved. Since the wider lateral section of the beam path surface of the second prism does not extend into the region between the first prism and the third prism, the arrangement of the support bar surface adds an additional element of tilting resistance in a transverse direction.

The support bar can be arranged on the side of the prism group that is arranged opposite the section of the second prism that is the least laterally widened. This arrangement offers a broader base and tilting resistance in the transverse direction of the second prism.

The support bar can have a contact surface for an abutting edge of the third prism. This simplifies and improves the alignment of the combination of the second prism and third prism in the mount in the longitudinal direction of the endoscope, or respectively endoscope shaft.

The support bar surface and at least one support surface for the at least one laterally wider section of the second prism can form a coherent, such as an L-shaped, T-shaped or H-shaped support surface. This reduces tilting in both directions when the second prism is pressed on the support surface during assembly.

When a lateral shoulder surface of the mount is provided for the lateral alignment of the second prism, a twisting of the set of prisms in the plane of the support surface is also avoided.

The object is also achieved by an endoscope with a variable viewing direction having an above-described prism holder arrangement.

The cited features, properties, and advantages of the disclosed embodiments also apply without restriction to the respective other subjects that relate to each other.

Further features will become apparent from the description of embodiments together with the claims and the included drawings. Embodiments disclosed herein can fulfill individual characteristics or a combination of several characteristics.

### BRIEF DESCRIPTION OF THE DRAWINGS

The embodiments are described below, without restricting the general idea, based on exemplary embodiments in reference to the drawings, whereby we expressly refer to the drawings with regard to the disclosure of all details that are not explained in greater detail in the text. In the figures:

FIG. 1 illustrates a schematic, perspective representation of an endoscope,

FIG. 2 illustrates a schematic side view of a known prism unit,

FIG. 3 illustrates a schematic plan view of a known prism unit,

FIG. 4 illustrates a schematic sectional representation of a prism holder arrangement for the prism unit of FIG. 2,

FIG. 5 illustrates a schematic perspective representation of the prism holder arrangement from FIG. 4,

FIG. 6 illustrates a schematic, perspective representation of the set of prisms of FIG. 2,

FIG. 7 illustrates a schematic perspective representation of a part of a set of prisms, and

FIG. 8 illustrates a schematic representation of a prism holder arrangement.

In the drawings, the same or similar types of elements and/or parts are provided with the same reference numbers so that a re-introduction is omitted.

### DETAILED DESCRIPTION

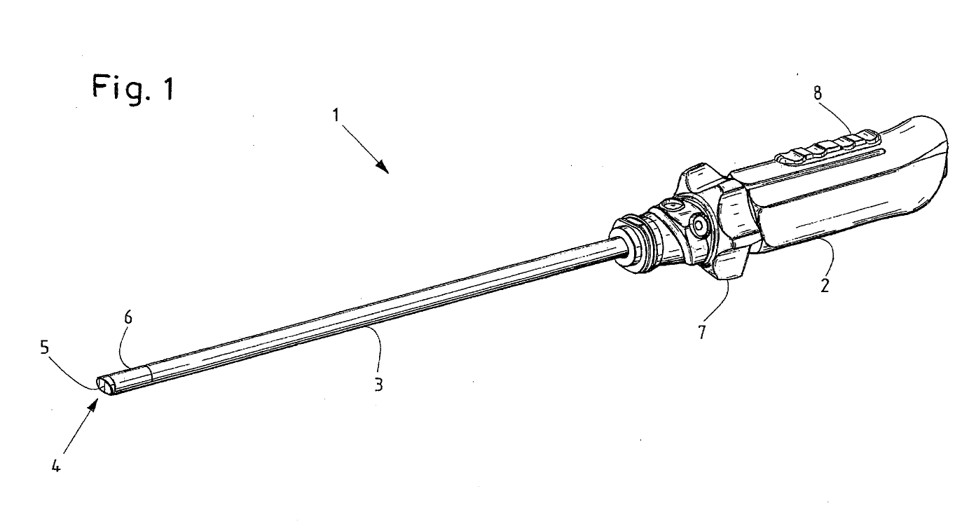

FIG. 1 shows a schematic perspective representation of an endoscope 1 with a proximal handle 2 and a rigid endoscope shaft 3. Arranged on the distal tip 4 of the endoscope shaft 3 is an inspection window 5, behind which a distal section 6 of the endoscope shaft is arranged that has a prism unit (not shown) and an image sensor unit (not shown). The endoscope 1 can be configured as a video endoscope.

The inspection window 5 at the distal tip 4 is configured curved and asymmetrical. The inspection window 5 is thereby configured to support a variable lateral viewing angle. A change in the viewing direction, i.e., a change in the azimuthal angle about the longitudinal axis of the endoscope shaft 3 is effectuated by a rotation of the handle 2 about the central rotational axis, or longitudinal axis, of the endoscope shaft 3. The jacket tube of the endoscope shaft 3 is connected to the handle. The prism unit (not shown) on the distal tip 4 also rotates with the rotation of the handle 2.

The handle 2 has a first control element configured as a rotary wheel 7, and a second control element configured as a slide 8.

To retain the horizontal position of the displayed image, the rotary wheel 7 is held while rotating the handle 2. This causes the image sensor within the interior of the endoscope shaft 3 to not follow the movement.

In order to change the viewing angle, i.e., the deviation of the viewing direction from the direct view, the slide 8 is moved. Moving the slide 8 in the distal direction causes for example an enlargement of the viewing angle, moving the slide 8 back in the proximal direction causes in this case a reduction of the viewing angle to a direct view. The actuation of the slide 8 is associated with a rotation of the image sensor in order to retain the horizontal position of the displayed image even when the prism unit is rotated.

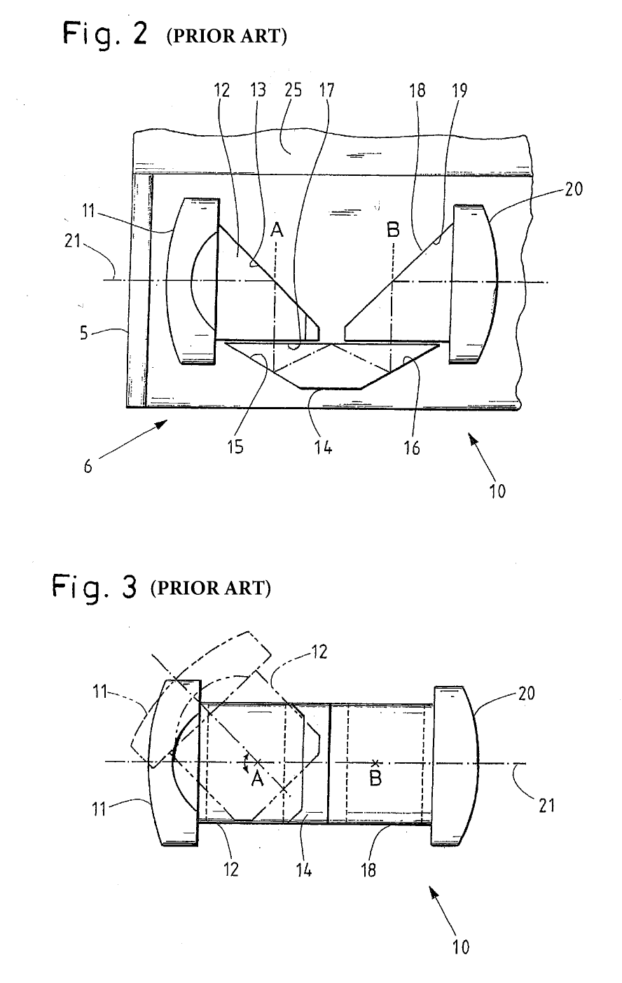

FIG. 2 schematically portrays a known prism unit 10 from the side. On the left side of the image, a light of a central beam path 21 depicted as a dot-dashed line enters an inspection window 5 and passes through an entrance lens 11 into a first, distal prism 12. The light contacts the mirror surface 13 and is reflected by total reflection or a mirror coating downwards toward a second prism 14 and a mirror surface 15 of the second prism. The mirror surface 15 has a sharp angle relative to the bottom side 17 of the second prism 14 so that the central beam path is first reflected by a central section of the bottom side 17 and from there to a second mirror surface 16 of the second prism 14. This second mirror surface 16 also has a sharp angle relative to the bottom side 17 so that the central beam path is contrastingly reflected upward (axis B). There, the light enters a third prism 18 with a mirror surface 19 through which the light of the central beam path 21 is then reflected centrally in a direction parallel to the longitudinal axis of the endoscope shaft 3, and exits through an exit lens 20 out of the prism unit 10. Above the prism unit 10, a part of an optical fiber bundle 25 is also depicted, by means of which light is conducted from proximal to the distal tip in order to illuminate an otherwise unilluminated surgical field.

The first prism 12 can be rotated about the perpendicular axis A in order to shift the lateral viewing angle. This also rotates the mirror surfaces 13 and 15 relative to each other so that the horizontal position of the image which is transferred in a proximal direction is altered about axis A upon a rotation of the first prism 12. This must be offset by a rotation of the image sensor or image sensors.

FIG. 3 shows the prism unit 10 from FIG. 2 in a schematic plan view. On the left side, it shows how the first prism 12 is arranged in a 0° viewing direction (solid line). Likewise, it shows in dashed lines that the first prism 12 has rotated together with the entrance lens 11 about the rotational axis A. In this case, the overlapping region is rotated between the mirror surfaces 13 of the first prism 12 and 15 in the second prism 14. The horizontal position is also correspondingly rotated.

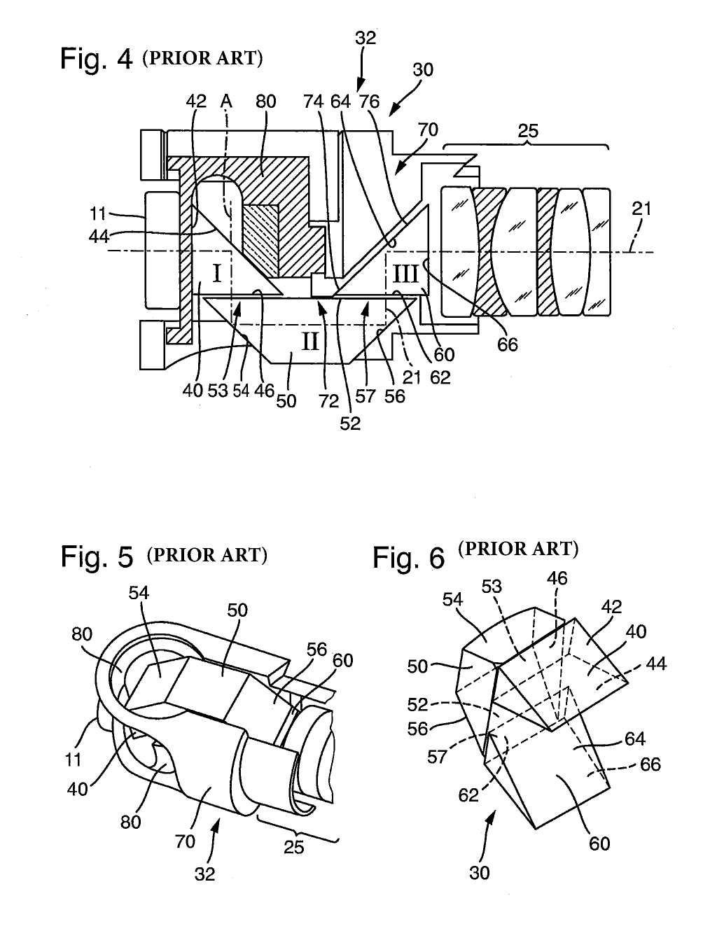

FIG. 4 shows a schematic sectional representation of a prism holder arrangement 32. The prism holder arrangement 32 holds a known set of prisms 30 with a first isosceles rectangular prism 40, a second prism 50, and a third isosceles and rectangular prism is 60. The width of the three prisms 40, 50 and 60 at least corresponds to the width of the beam path of which the central beam path 21 is depicted.

Light enters through the entrance lens 11 and passes through a light entry surface 42 into the first prism 40. The incoming light is reflected from the mirror surface 44, for example by total reflection, and passes through the light exit surface 46 into the second prism 50. In so doing, it passes through a light entry region 53 of the beam path surface 52 of the second prism 50 and contacts the first mirror surface 54. The light is reflected by the first mirror surface 54 and then by the second mirror surface 56 and exits the light exit region 57 of the beam path surface 52 of the second prism 50 and enters the third prism 60 through the light entry surface 62. The light is in turn reflected by the mirror surface 64 of the third prism 60 and exits the third prism 60 through the light exit surface 66 and enters a rearward lens group 25 with a plurality of lenses and lens mounts that are identified by hatching. An image sensor, a pair of image sensors, or a series of rod lens relay sets which are not shown in FIG. 4 can follow the exit of the lens group 25.

It is also possible to configure the second prism 50 flatter and provide a third mirror coating on the beam path surface 52 between the light entry region 53 and light exit region 57. This version is shown for example in FIG. 2.

The optical elements are held and borne in a mount 70, wherein the first prism 40 and entrance lens 11 are rotatably arranged in, or respectively on a swivel prism holder 80 about a rotational axis A. The rotational axis A coincides with a section of the deflected central beam path 21. The swivel prism mount 80 is rotatably mounted about the same rotational axis A in the mount 70. The swivel prism holder 80 is depicted with hatching.

In the region of the central beam path surface 52 and mirror surface 64 of the second prism 50 and third prism 60, the mount 70 has a structure that constitutes a support bar surface 72 and angled contact surface 74. The support bar surface 72 is comparatively narrow and hence susceptible to tilting by the combination of the second prism 50 and third prism 60. Since the angled contact surface 74 terminates in the top region and leaves space for a cavity 76, the angled contact surface 74 also does not offer any tilting resistance.

FIG. 5 shows a schematic perspective representation of the prism holder arrangement 32 from FIG. 4. In the view of the bottom side of the prism holder arrangement 32, the bottom side of the second prism 50 with the first mirror surface 54 which is arranged distally can be seen along with the second mirror surface 56 in the holder 70. Proximally, a small section of the light entry surface of the third prism 60 is discernible. Distally, a small section of the first prism 40 is furthermore discernible which is mostly covered by the holder 70. Furthermore, the swivel prism holder 80 which is depicted with hatching is partially discernible as well as the entrance lens 11. In the distal region, the holder 70 has a cylindrical cavity in which the also cylindrical swivel prism holder 80 is arranged.

FIG. 6 shows a schematic, perspective representation of the set of prisms from FIG. 4, wherein the most important edge lines of the prisms 40, 50 and 60 are depicted. Such edge lines which lie behind a layer of glass are dashed. The reference numbers correspond to those from FIG. 4. It can be seen that the second prism 50 has the same width as the first prism 40 and the third prism 60. At their respective distal or respectively proximal ends, both the first prism 40 and the third prism 60 extend slightly beyond the second prism 50. Between the first prism 40 and third prism 60, there is a narrow region of the beam path surface 52 of the second prism 50 that is not covered by one of the two other prisms. Only this region is available for the support surface and tilt resistance.

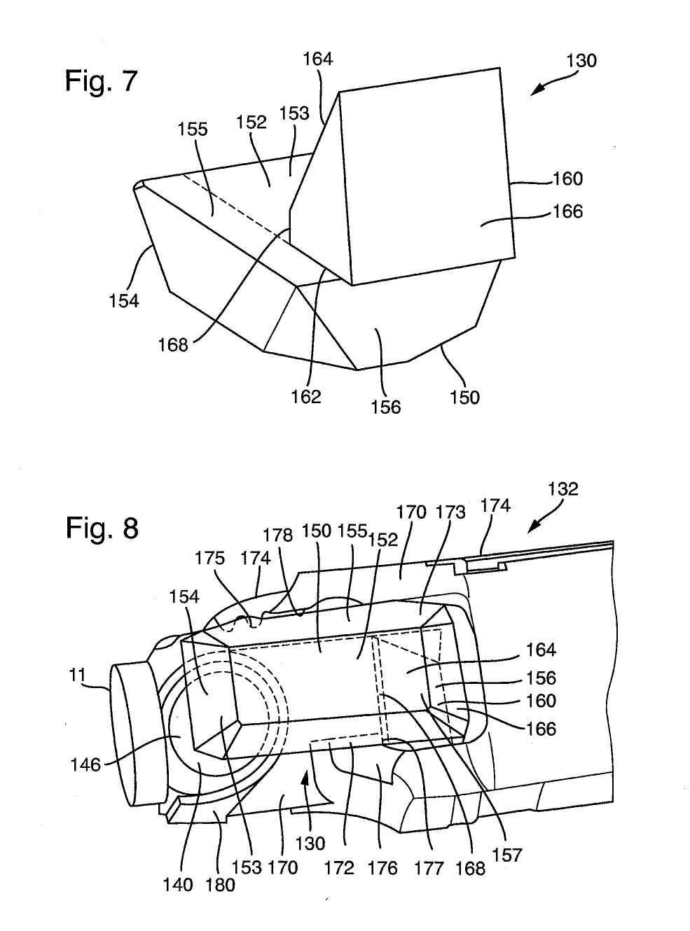

FIG. 7 shows a schematic perspective representation of a portion of a set of prisms 130 for use in the endoscope of FIG. 1. In this case, only the second prism 150 and third prism 160 are depicted. For reasons of clarity, although part of the set of prisms 130, the first prism 140 is omitted. The third prism 160 is glued to the second prism 150. The third prism 160 is narrower than the second prism 150. On the left side in FIG. 7, the beam path surface 152 has a lateral wider section 155 that is not covered by the third prism 160. The extension of the side edge of the third prism 160 is dotted to render the lateral wider section 155 discernible over the entire length.

The light entry region 153 on the beam path surface 152, the first mirror surface 154 and second mirror surface 156 of the second prism 150 are also depicted in FIG. 7. In order to save installation space, some edges and corners of the second prism 150 have been chamfered.

With the third prism 160, the light exit surface 166 and light entry surface 162 facing the beam path surface 152 of the second prism 150 are identified along with the mirror surface 164. The mirror surface 164 terminates before the light entry surface 162 in an abutting edge 168. This is arranged perpendicular to the light entry surface 162 of the third prism 160 and to the beam path surface 152 of the second prism 150 and can serve to align the combination of the second prism 150 and third prism 160 in a mount.

FIG. 8 furthermore shows a schematic perspective representation of a prism holder arrangement 132. This is also a perspective view of the bottom side of a corresponding mount 170 that is arranged distally on the endoscope shaft and has distal structures for holding the set of prisms 130 according to FIG. 7, i.e., at the head end. Distally, there is an entrance lens 11 and directly afterward a substantially cylindrical swivel prism holder 180 with an implied swivel prism as a first prism 140. In the present case according to FIG. 7, the swivel prism itself is cylindrical, wherein a light entry surface (not shown) is enclosed in the cylindrical body as a flat surface parallel to the cylinder axis. This is oriented toward the entrance lens 11. The mirror surface is cut at a 45° angle relative to the cylinder axis. The light exit surface is identified by reference number 146.

A corresponding cylindrical swivel prism is disclosed in parallel German application No. 10 2014 202 612.1 by the applicant which has the same filing date as the present application and which corresponds to U.S. application Ser. No. 15/233,160 filed on Aug. 10, 2016 (attorney Docket Number 33489Z), the contents of which is incorporated herein by reference.

The second prism 150 is wider on the side shown in FIG. 8 at the top with a section 155 that extends beyond the first and third prisms 140, 160. This wider section 155 serves to resist tilting in that it lies on a side support surface 173 of the holder 170. A support bar 176 is also shown on the opposite side on the support bar surface 172 of which the second prism 150 partially lies. At its proximal end, the support bar 176 also has a contact edge (without a reference number) against which the abutting edge 168 of the third prism 160 lies. Another anti-twist protection is provided by a lateral shoulder surface 178 against which the second prism 150 presses.

Furthermore, a shifting element 174 that can shift along a longitudinal axis is depicted that, by means of teeth elements 175, converts longitudinal axial shifts by the proximal handle into a rotary movement of the swivel prism holder 180 which has complementary teeth elements (not shown).

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### REFERENCE NUMBER LIST

1 Endoscope

2 Handle

3 Endoscope shaft

4 Distal tip

5 Viewing window

6 Distal section

7 Rotary wheel

8 Sliding switch

9 Jacket tube

10 Prism unit

11 Entrance lens

12 Swivel prism

13 Mirror surface

14 Second prism

15, 16 Mirror surface

17 Bottom side

18 Third prism

19 Mirror surface

20 Exit lens

21 Central beam path

25 Rearward lens group

30 Set of prisms

32 Prism holder arrangement

40 First prism

42 Light entry surface

44 Mirror surface

46 Light exit surface

50 Second prism

52 Beam path surface

53 Light entry region

54 First mirror surface

56 Second mirror surface

57 Light exit region

60 Third prism

62 Light entry surface

64 Mirror surface

66 Light exit surface

70 Mount

72 Support bar surface

74 Angled contact surface

76 Cavity

80 Swivel prism holder

130 Set of prisms

132 Prism holder arrangement

140 First prism

146 Light exit surface

150 Second prism

152 Beam path surface

153 Light entry region

154 First mirror surface

155 Lateral wider section

156 Second mirror surface

157 Light exit region

160 Third prism

162 Light entry surface

164 Mirror surface

166 Light exit surface

168 Abutting edge

170 Mount

172 Support bar surface

173 Lateral support surface

174 Shifting element

175 Tooth element

176 Support bar

177 Contact surface

178 Lateral shoulder surface

180 Swivel prism holder

What is claimed is:

1. A set of prisms for an endoscope with a variable viewing direction, the set of prisms comprising:
a first prism,
a second prism; and
a third prism;
wherein the first, second and third prisms together define a beam path passing through the first, second and third prisms;
the first prism and the third prism each have a light entrance surface, a mirror surface and a light exit surface, and the second prism has a single beam path surface having a light entrance region and a light exit region and at least two mirror surfaces,
the first prism can be pivoted with respect to the second prism about an axis of rotation that runs through the mirror surface of the first prism and one of the at least two mirror surfaces of the second prism, and the third prism is arranged in an immovable manner with respect to the second prism, and
the second prism is configured at least sectionally wider than the first prism and/or the third prism transversally to a course of the beam path such that at least one lateral wider section of the single beam path surface of the second prism extends on at least one side beyond the light entrance surface of the third prism.

2. The set of prisms according to claim 1, wherein the at least one lateral wider section of the single beam path surface of the second prism extends on at least one side beyond the light exit surface of the first prism in a 0° viewing direction of the set of prisms.

3. The set of prisms according to claim 1, wherein the third prism has an abutting edge on a side of the third prism facing the first prism.

4. The set of prisms according to claim 1, wherein the third prism is glued to the second prism.

5. A prism holder arrangement for an endoscope, the prism holder arrangement comprising the set of prisms according to claim 1 with a variable viewing direction, and a mount for the set of prisms, wherein the mount has at least one support surface for the at least one lateral wider section of the single beam path surface of the second prism which, in the longitudinal direction of the endoscope, extends longer than a minimum distance between the first prism and the third prism.

6. The prism holder arrangement according to claim 5, wherein the mount further comprises a support bar with a support bar surface, wherein in a mounted state, a section of the single beam path surface of the second prism lies on the support bar surface which is arranged between the first prism and third prism.

7. The prism holder arrangement according to claim 6, wherein the support bar is arranged on a side of the prism group that is arranged opposite the at least one lateral wider section of the second prism.

8. The prism holder arrangement according to claim 6, wherein the support bar has a contact surface for an abutting edge of the third prism.

9. The prism holder arrangement according to claim 6, wherein the support bar surface and at least one support surface for the at least one lateral wider section of the second prism form a coherent support surface.

10. The prism holder arrangement according to claim 6, wherein a lateral shoulder surface of the mount is provided for the lateral alignment of the second prism.

11. An endoscope with a variable viewing direction comprising a prism holder arrangement according to claim 5.
